(19) United States
(12) Patent Application Publication
(10) Pub. No.: US 2023/0145301 A1
(43) Pub. Date: May 11, 2023

(54) MEDICAL INSTRUMENT SET, DELIVERY SYSTEM, AND EMBOLIC DEVICE DELIVERY MEDICAL SYSTEM

(71) Applicant: TERUMO KABUSHIKI KAISHA, Tokyo (JP)

(72) Inventors: Hideaki SHIBATA, Hadano-shi (JP); Eri IKUNO, Hadano-shi (JP)

(73) Assignee: TERUMO KABUSHIKI KAISHA, Tokyo (JP)

(21) Appl. No.: 18/154,129

(22) Filed: Jan. 13, 2023

Related U.S. Application Data
(63) Continuation of application No. PCT/JP2021/026419, filed on Jul. 14, 2021.

(30) Foreign Application Priority Data
Jul. 16, 2020 (JP) 2020-122112

(51) Int. Cl. A61B 17/12

(52) U.S. Cl. CPC A61B 17/1219; A61B 17/12118; A61B 17/12113; A61B 2017/12054

(57) ABSTRACT

A medical instrument set includes a embolic device loading catheter that includes a catheter body including a loading lumen having an open distal end, and a proximal hub including an insertion passage communicating with a proximal end of the loading lumen, and in which an embolic device is loaded in the loading lumen; and a delivery pusher that includes an elongated pusher body insertable into the loading lumen through the insertion passage of the proximal hub of the embolic device loading catheter. The catheter body includes a distal connection portion attachable to and detachable from a sheath hub on a distal side of the catheter body. The pusher body has a body length longer than a distance from a proximal end of the insertion passage of the proximal hub to a distal end opening of a sheath communicating with a sheath lumen of a delivery catheter.

### CROSS-REFERENCES TO RELATED APPLICATIONS

This application is a continuation of International Application No. PCT/JP2021/026419 filed on Jul. 14, 2021, which claims priority to Japanese Application No. 2020-122112 filed on Jul. 16, 2020, the entire content of both of which is incorporated herein by reference.

### TECHNOLOGICAL FIELD

The present disclosure generally relates to a medical instrument set, a delivery system, and an embolic device delivery medical system.

### BACKGROUND DISCUSSION

There is currently no drug treatments that can prevent an aneurysm (aortic aneurysm) generated in an aorta of a patient from increasing in diameter and rupturing. Surgical treatment (surgery) is generally performed for an aneurysm having a diameter that has a risk of rupturing. Further, in a surgery of the aortic aneurysm of the related art, artificial blood vessel replacement surgery in which an artificial blood vessel is transplanted by laparotomy or thoracotomy has been used, and in recent years, application of lower invasive stent graft interpolation (endovascular aneurysm repair: EVAR) has been rapidly expanding.

For example, in stent graft interpolation for an abdominal aortic aneurysm (AAA), a catheter having a stent graft at a distal end of the stent graft is inserted from a peripheral blood vessel of the patient, and the stent graft is expanded and indwells in an aneurysm lesion, thereby blocking a blood flow to the aneurysm and preventing the aneurysm from rupturing.

In general, a stent graft used in the stent graft interpolation has a structure in which two types of members, that is, a “main body portion” including bifurcated portions bifurcated into a substantially Y-shape and “leg portions” attached to the bifurcated portions and attached to a right iliac artery and a left iliac artery, respectively, are assembled.

Therefore, in the stent graft interpolation, so-called “endoleak” in which the blood flow remains in the aneurysm may occur due to blood leakage from a periphery of the stent graft due to insufficient adhesion of the interpolated stent graft, backflow of blood from a thin blood vessel (side branch blood vessel) branched from the aneurysm, or the like. In this case, since pressure is applied to a wall of the aneurysm by the blood flow entering the aneurysm, there is a potential risk of rupture of the aneurysm.

U.S. Pat. No. 9,561,096 discloses a device including a catheter capable of holding a compressed relatively elongated sponge (embolic device) in a lumen of the device and a plunger for pushing out the embolic device held in the catheter into an aneurysm filled with blood in order to block blood flow remaining in an aortic aneurysm due to endoleak. Since the sponge used in this device expands immediately when exposed to the blood, the sponge is pushed out into the aneurysm and expands when absorbing the blood in the aneurysm, and the sponge indwells in the aneurysm in this state to block the blood flow and prevent rupture.

In endoleak embolization including the technique disclosed in U.S. Pat. No. 9,561,096, the following “direct insertion method” and “indirect insertion method” are conceivable as embolic device delivery methods for delivering an embolic device into an aneurysm through a catheter.

The “direct insertion method” is a delivery method including: a procedure for inserting a first catheter in which an embolic device is loaded into a sheath of a second catheter indwelling in a body lumen (procedure A1); a procedure for inserting a delivery pusher into the first catheter while a distal end of the first catheter reaches the aneurysm to deliver the embolic device into the aneurysm (procedure A2); and a procedure for removing the first catheter and the delivery pusher from the second catheter after the embolic device indwells in the aneurysm (procedure A3). In the direct insertion method, the embolic device is pushed out into the aneurysm and indwells in the aneurysm by inserting and pushing out the delivery pusher into the first catheter.

The “indirect insertion method” is a delivery method including: a procedure for inserting part of the distal end of the first catheter loaded with the embolic device into the sheath of the second catheter indwelling in the body lumen (procedure B1); a procedure for inserting a loading pusher into the first catheter to transfer the embolic device to the second catheter (procedure B2); a procedure for removing the loading pusher and the first catheter from the second catheter (procedure B3); a procedure for inserting the delivery pusher into the second catheter to deliver the embolic device into the aneurysm (procedure B4); and a procedure for removing the delivery pusher from the second catheter after the embolic device indwells in the aneurysm (procedure B5). In the indirect insertion method, the embolic device is moved from the first catheter to the second catheter by the loading pusher, and is pushed out into the aneurysm and indwelling in the aneurysm by inserting and pushing out the delivery pusher into the second catheter.

However, these two delivery methods have the following problems (problems 1 to 3), particularly in terms of the complexity of the procedures and procedure time.

(Problem 1) In the direct insertion method, the first catheter requires flexibility and kink resistance such that the first catheter can be inserted following a shape of the meandering blood vessel. In order to fulfill these functions, it is necessary to make a flexible catheter body, and there is a risk that the loaded embolic device may be damaged during packaging or unpacking. When the embolic device indwells in the aneurysm, both the first catheter and the delivery pusher should be inserted into the aneurysm along a meandering flow path of the blood vessel, which can increase the difficulty of the procedures.

(Problem 2) In the indirect insertion method, since the number of procedures is larger than that in the direct insertion method, and procedures such as insertion and removal of a device are complicated, the procedure time may be longer.

(Problem 3) Both the direct insertion method and the indirect insertion method are methods for pushing out the embolic device into the aneurysm in a state where at least a part of the first catheter to which the embolic device is loaded is inserted into the second catheter. Accordingly, an outer diameter of the first catheter is smaller than an inner diameter of the second catheter, and an outer diameter of the embolic device is smaller than an inner diameter of the first catheter. Therefore, in order to make a required amount of small-diameter embolic device indwell in the aneurysm, the number of inserted embolic device inevitably increases, and the procedure time may be relatively long.

### SUMMARY

A medical instrument set, a delivery system, and an embolic device delivery medical system are disclosed, which are capable of shortening procedure time by simplifying the procedure and reducing the number of steps of the procedure.

A medical instrument set according to an embodiment is a medical instrument set for delivering an embolic device into an aneurysm via a delivery catheter. The delivery catheter includes a sheath that includes a sheath lumen and a distal end opening that communicates with the sheath lumen, and a sheath hub that includes a communication passage communicating with a proximal end of the sheath lumen and is provided on a proximal side of the sheath. The medical instrument set includes: an embolic device loading catheter that includes a catheter body including a loading lumen loaded with the embolic device and having an open distal end, and a proximal hub including an insertion passage communicating with a proximal end of the loading lumen; and a delivery pusher that includes an elongated pusher body insertable into the loading lumen through the insertion passage of the proximal hub of the embolic device loading catheter. The catheter body includes a distal connection portion attachable to and detachable from the sheath hub of the delivery catheter on a distal side of the catheter body. The pusher body has a body length longer than a distance from a proximal end of the insertion passage of the proximal hub to the distal end opening of the sheath of the delivery catheter in a connected state where the distal connection portion of the catheter body is connected to the sheath hub of the delivery catheter, and when the pusher body is inserted from the insertion passage of the proximal hub in the connected state, the embolic device is passed through the sheath lumen of the delivery catheter and pushed out from the distal end opening of the delivery catheter into the aneurysm.

A delivery system according to an embodiment includes the medical instrument set and the delivery catheter, and is configured such that, in the connected state, the embolic device is passed through the sheath lumen of the delivery catheter by the delivery pusher inserted from the insertion passage of the proximal hub and is pushed out from the distal end opening of the delivery catheter into the aneurysm.

An embolic device delivery medical system according to an embodiment includes: the delivery system; and an elongated insertion assisting member to be assembled into the sheath lumen of the delivery catheter in order to assist delivery of the delivery catheter into an aneurysm. The insertion assisting member includes a guide wire lumen that penetrates from a distal end to a proximal end and has a diameter smaller than that of the sheath lumen of the delivery catheter.

An embolic device delivery medical system according to an embodiment includes: a delivery catheter, the delivery catheter including a sheath that includes a sheath lumen and a distal end opening that communicates with the sheath lumen, and a sheath hub that includes a communication passage communicating with a proximal end of the sheath lumen and is provided on a proximal side of the sheath; an embolic device loading catheter that includes a catheter body including a loading lumen configured to be loaded with the embolic device and having an open distal end, and a proximal hub including an insertion passage communicating with a proximal end of the loading lumen; a delivery pusher that includes an elongated pusher body insertable into the loading lumen through the insertion passage of the proximal hub of the embolic device loading catheter; the embolic device loading catheter including a distal connection portion configured to be attachable to and detachable from a sheath hub of a delivery catheter at a distal portion of the catheter body; the pusher body having a body length longer than a distance from a proximal end of the insertion passage of the proximal hub to the distal end opening of the sheath of the delivery catheter in a connected state where the distal connection portion of the catheter body is connected to the sheath hub of the delivery catheter, and when the pusher body is inserted from the insertion passage of the proximal hub in the connected state, the embolic device is configured to pass through the sheath lumen of the delivery catheter and to be pushed out from the distal end opening of the delivery catheter into the aneurysm; and an elongated insertion assisting member configured to be assembled into the sheath lumen of the delivery catheter in order to assist delivery of the delivery catheter into the aneurysm, and wherein the insertion assisting member includes a guide wire lumen that penetrates from a distal end to a proximal end and has a diameter smaller than that of the sheath lumen of the delivery catheter.

A method according to an embodiment includes: introducing a distal portion of a delivery catheter into a living body and positioning the distal end of the delivery catheter in an aneurysm in the living body, the delivery catheter including a sheath that includes a sheath lumen and a distal end opening that communicates with the sheath lumen, and a sheath hub that includes a communication passage communicating with a proximal end of the sheath lumen and is provided on a proximal side of the sheath; connecting a distal portion of an embolic device loading catheter to the proximal end of the delivery catheter, the embolic device loading catheter including a catheter body including a loading lumen loaded with an embolic device and having an open distal end, and a proximal hub including an insertion passage communicating with a proximal end of the loading lumen; introducing a distal end of a delivery pusher into the embolic device loading catheter by way of the proximal end of the loading lumen, the delivery pusher including an elongated pusher body insertable into the loading lumen through the insertion passage of the proximal hub of the embolic device loading catheter, and wherein the pusher body has a body length longer than a distance from a proximal end of the insertion passage of the proximal hub of the embolic device loading catheter to the distal end opening of the sheath of the delivery catheter when the distal portion of the catheter body is connected to the proximal end of the delivery catheter; and moving the delivery pusher in a forward direction to push the embolic device out of the embolic loading catheter into the sheath lumen of the delivery catheter and out of the sheath lumen of the delivery catheter into the aneurysm.

According to at least one embodiment of the disclosure, it is possible to shorten the procedure time by simplifying the procedure and reducing the number of steps associated with the procedure.

### BRIEF DESCRIPTION OF THE DRAWINGS

FIG. 1 is a diagram showing configurations of a medical instrument set and a delivery set according to the present embodiment.

FIG. 2 is a diagram showing a configuration of an embolic device delivery medical system according to the present embodiment.

FIG. 3A is a schematic cross-sectional view showing one configuration example of an engagement portion of the medical instrument set.

FIG. 3B is a schematic cross-sectional view showing an engaged state of the engagement portion in FIG. 3A.

FIG. 4A is a schematic cross-sectional view showing another configuration example of the engagement portion of the medical instrument set.

FIG. 4B is a schematic cross-sectional view showing an engaged state of the engagement portion in FIG. 4A.

FIG. 5 is a schematic cross-sectional view showing a connected state between an embolic device loading catheter and a delivery catheter.

FIG. 6A is a partially enlarged view of a vicinity of a handle portion of a delivery pusher.

FIG. 6B is a partially enlarged view showing an inserted state of the delivery pusher.

FIG. 7A is an operation example of the embolic device delivery medical system according to the present embodiment, showing a state where the delivery catheter is delivered into an aneurysm.

FIG. 7B is an operation example of the embolic device delivery medical system according to the present embodiment, showing a state where a stent graft is deployed in the aneurysm.

FIG. 7C is an operation example of the embolic device delivery medical system according to the present embodiment, showing a state before the embolic device loading catheter is connected to the delivery catheter.

FIG. 7D is an operation example of the embolic device delivery medical system according to the present embodiment, showing a state where the embolic device loading catheter is connected to the delivery catheter.

FIG. 7E is an operation example of the embolic device delivery medical system according to the present embodiment, showing a state where the delivery pusher is being inserted into the embolic device loading catheter.

FIG. 7F is an operation example of the embolic device delivery medical system according to the present embodiment, showing a state where the embolic device is pushed out into the aneurysm by the delivery pusher.

FIG. 7G is an operation example of the embolic device delivery medical system according to the present embodiment, showing a state where the embolic device loading catheter is detached from the delivery catheter.

FIG. 8A is a modification of the embolic device loading catheter according to the present embodiment, showing a state before the engagement portion is engaged.

FIG. 8B is a modification of the embolic device loading catheter according to the present embodiment, showing a state after the engagement portion is engaged.

### DETAILED DESCRIPTION

Set forth below with reference to the accompanying drawings is a detailed description of embodiments of a medical instrument set, a delivery system, and an embolic device delivery medical system. Note that since embodiments described below are preferred specific examples of the present disclosure, although various technically preferable limitations are given, the scope of the present disclosure is not limited to the embodiments unless otherwise specified in the following descriptions. In the drawings, the same or corresponding parts are denoted by the same reference numerals. In the description of the present embodiment, the description of the same or corresponding parts will be omitted or simplified as appropriate.

Further, in the accompanying drawings, for convenience of illustration and understanding, a scale, an aspect ratio, a shape, and the like may be changed from actual ones and may be schematically expressed as appropriate, and the drawings are just examples and do not limit the interpretation of the disclosure.

In the present specification, in an operation direction of each part constituting an embolic device delivery medical system 300, for example, a side on which an embolic device 10 is to be conveyed into an aneurysm in a direction along an axial direction of a delivery catheter 40 is referred to as a “distal side (or a distal portion)”, and a side that is located on a side opposite to the distal side in the axial direction and on which a surgeon performs an operation, for example, with his/her hands (a side from which the delivery catheter 40 is removed) is referred to as a “proximal side (or a proximal portion)”. In the present specification, a “distal end” means a certain range in the axial direction including a most distal end, and a “proximal end” means a certain range in the axial direction including a most proximal end.

A medical instrument set 100, a delivery system 200, and the embolic device delivery medical system 300 according to the present embodiment may be applied to endoleak embolization for stent graft interpolation of an abdominal aortic aneurysm (AAA), which is an example of a treatment method for preventing rupture of an aneurysm generated in a blood vessel. In addition, the treatment method in which the medical instrument set 100, the delivery system 200, and the embolic device delivery medical system 300 according to the present embodiment are used is not limited to the endoleak embolization, and can also be applicable to other intervention treatment methods for preventing the rupture of the aneurysm generated in the blood vessel.

Configuration

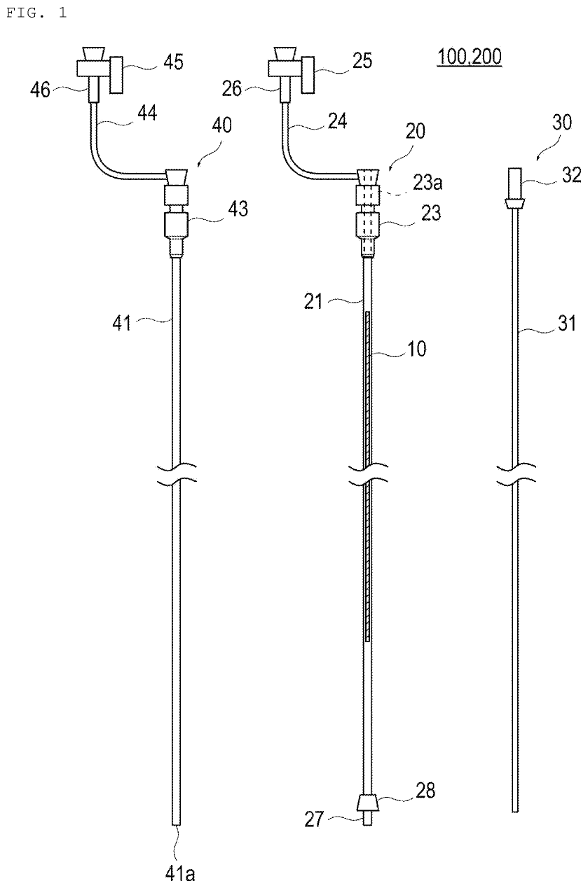

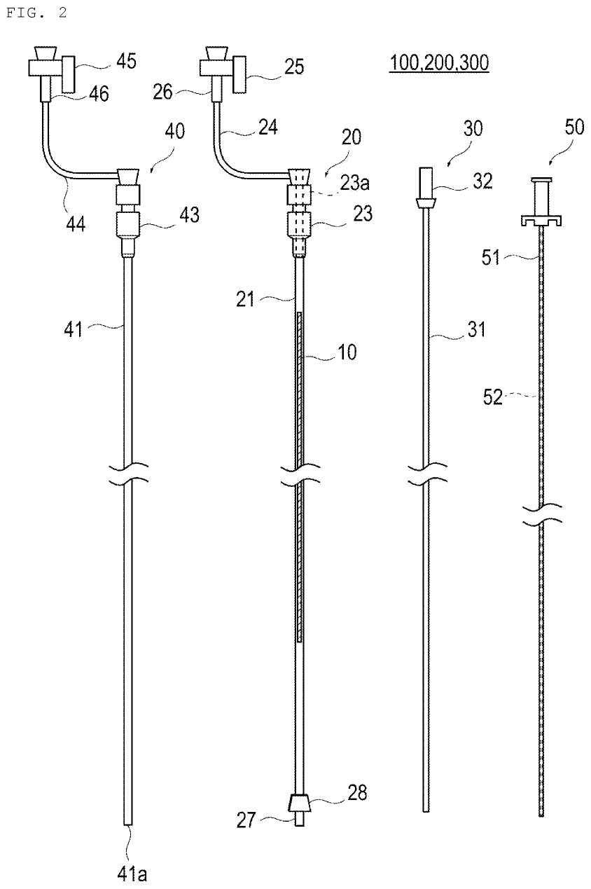

Next, configurations of the medical instrument set 100, the delivery system 200, and the embolic device delivery medical system 300 according to the present embodiment will be described. FIG. 1 shows each device constituting the medical instrument set 100 and the delivery system 200 according to the present embodiment, and FIG. 2 shows each device constituting the embolic device delivery medical system 300 according to the present embodiment.

First, the embolic device 10 used in the medical instrument set 100, the delivery system 200, and the embolic device delivery medical system 300 according to the present embodiment will be described.

Embolic Device

The embolic device 10 indwells in an aneurysm such as an aortic aneurysm generated in the blood vessel and expands by absorbing a liquid containing blood flowing into the aneurysm. The embolic device 10 is loaded into an embolic device loading catheter 20, and with the embolic device loading catheter 20 connected to the delivery catheter 40, the embolic device 10 is pushed out by a delivery pusher 30 and indwells in the aneurysm.

The embolic device 10 can be an elongated fibrous linear body made of an expandable material (polymer material (water-absorbing gel material) or the like) that expands by contacting with an aqueous liquid containing the blood under a physiological condition. That is, in this embodiment, the embolic device 10 is a swellable embolic material. The embolic device 10 is an elongated umbilical member having a substantially circular cross-sectional shape in a direction orthogonal to a longitudinal direction, and is relatively fragile before expansion in which the embolic device 10 indwells in the aneurysm. The cross-sectional shape of the embolic device 10 is not particularly limited, and may be a polygonal shape in addition to the substantially circular shape.

Here, the “physiological condition” means a condition having at least one environmental characteristic in a body or on a body surface of a mammal (for example, humans). Such a characteristic includes an isotonic environment, a pH buffering environment, an aqueous environment, a pH near neutral (about 7), or a combination of the isotonic environment, the pH buffering environment, the aqueous environment, and the pH near neutral. The “aqueous liquid” includes, for example, body fluids of a mammal (for example, humans) such as isotonic fluid, water, blood, spinal fluid, plasma, serum, glass body fluid, and urine. An outer diameter of the embolic device 10 may be accommodated within inner diameters of the embolic device loading catheter 20 and the delivery catheter 40, and may be substantially equal to the inner diameters of these catheters. A total length of the embolic device 10 is not particularly limited, and can be appropriately set depending on factors such as a size of the aneurysm to indwell in consideration of ease of loading and shortening of procedure time.

A constituent material from which the embolic device 10 may be fabricated is not particularly limited as long as the constituent material is a material that expands by absorbing at least a liquid such as blood, and has no (or extremely low) harmful effect on a human body even in a state of indwelling in the aneurysm. In addition, the embolic device 10 may be added or fabricated with a visualization material whose existing position in a living body can be confirmed by a confirmation method such as an X-ray, a fluorescent X-ray, an ultrasound, a fluorescence method, an infrared ray, or an ultraviolet ray.

Medical Instrument Set

Next, the configuration of the medical instrument set 100 according to the present embodiment will be described. As shown in FIG. 1, the medical instrument set 100 according to the present embodiment includes the embolic device loading catheter 20 and the delivery pusher 30.

Embolic Device Loading Catheter

The embolic device loading catheter 20 can include an elongated catheter body 21 in which a loading lumen 22 is provided, a proximal hub 23 provided on a proximal side of the catheter body 21, and a flexible tube 24 having one end connected to a proximal side of the proximal hub 23 and the other end connected to a port 26 of a three-way stopcock 25.

The catheter body 21 can be a tubular member formed with a hole (the loading lumen 22) extending from an opening portion on a distal side of the catheter body 21 to an opening portion on the proximal side of the catheter body 21 along an axial direction. A length of the catheter body 21 in an extending direction can be appropriately defined, and it is sufficient that the catheter body 21 has a length at least capable of accommodating the embolic device 10.

An inner diameter of the loading lumen 22 is designed to be substantially equal to an inner diameter of a sheath lumen 42 of the delivery catheter 40. Accordingly, the outer diameter of the embolic device 10 is substantially equal to inner diameters of the embolic device loading catheter 20 and the delivery catheter 40, and it is not necessary to reduce a diameter size of the embolic device 10 as in a direct insertion method and an indirect insertion method which can be assumed as a delivery method for the embolic device 10. Therefore, when a required amount of the embolic device 10 indwells in the aneurysm, the number of inserted embolic devices 10 can be reduced, and procedure time can be shortened.

The embolic device loading catheter 20 is mainly provided in a state where the embolic device 10 is loaded in advance, and the embolic device 10 to be loaded into the catheter body 21 may be loaded into the catheter body 21 by the surgeon or the like grasping the embolic device 10. As a method of loading the embolic device 10, the surgeon can grip the embolic device 10 and insert the embolic device 10 from a distal connection portion 27 side or a proximal hub 23 side of the embolic device loading catheter 20.

The catheter body 21 is connected (or mounted) by being engaged with a sheath hub 43 of the delivery catheter 40 in a state where the embolic device 10 is accommodated by an engagement portion 60, which will be described later. In this connected state, the delivery pusher 30 is inserted from the proximal hub 23 to push the loaded embolic device 10 toward the delivery catheter 40.

Since the embolic device loading catheter 20 is not inserted into a sheath of the delivery catheter 40 as in the direct insertion method, the embolic device loading catheter 20 does not require flexibility to follow a bent shape of a body lumen during insertion. Therefore, a constituent material from which the catheter body 21 may be fabricated is not particularly limited as long as the constituent material is a material that is at least more rigid than the delivery catheter 40 and provides an appropriate degree of hardness to prevent breakage of the loaded embolic device 10 during packaging or the like. As examples of the constituent material for the catheter body 21, a resin material such as a polymer material including polyolefins (for example, polyethylene, polypropylene, polybutene, an ethylene-propylene copolymer, an ethylene-vinyl acetate copolymer, an ionomer, or a mixture of two or more types thereof), a polyolefin elastomer, a crosslinked polyolefin, polyvinyl chloride, polyamide, a polyamide elastomer, polyester, polyester elastomer, polyurethane, polyurethane elastomer, a fluorous resin, polycarbonate, polystyrene, polyacetal, polyimide, polyetherimide, and aromatic polyether ketone, or a mixture thereof, and a metal material such as a shape memory alloy, stainless steel, tantalum, titanium, platinum, gold, or tungsten can be suitably used.

Since it is sufficient that the catheter body 21 is more rigid than a sheath 41 from the viewpoint of preventing breakage of the embolic device 10, in addition to making or fabricating the material for the catheter body 21 rigid, the catheter body 21 may be configured such that the catheter body 21 has a thickness so as not to be kinked when the same material as that of the sheath 41 is used. When the thickness is variable, an outer diameter of the catheter main body 21 is larger than an outer diameter of the sheath 41, but since the embolic device loading catheter 20 is connected to the delivery catheter 40 via the engagement portion 60, there is no particular problem.

The proximal hub 23 is an intermediate member that includes an insertion passage 23a (lumen) that allows communication between the loading lumen 22 of the catheter body 21 and the tube 24, and allows a fluid (such as saline(or saline solution) for priming) flowing from the three-way stopcock 25 to flow to the catheter body 21 via the tube 24. The embolic device 10 loaded into the loading lumen 22 is pushed toward the delivery catheter 40 by the delivery pusher 30 being inserted into the loading lumen 22 via the insertion passage 23a of the proximal hub 23.

A constituent material from which the proximal hub 23 may be fabricated is not particularly limited as long as the constituent material is a hard material such as a hard resin. As examples of the constituent material for the proximal hub 23, polyolefins such as polyethylene or polypropylene, polyamide, polycarbonate, or polystyrene can be suitably used.

A hemostasis valve is attached into the proximal side of the proximal hub 23. As the hemostasis valve, for example, a substantially elliptical film-shaped (disk-shaped) valve body made of silicone rubber, latex rubber, butyl rubber, or isoprene rubber which is an elastic member may be used.

The one end of the tube 24 is interlocked to the proximal side of the proximal hub 23, and the other end is interlocked to the port 26 of the three-way stopcock 25. The tube 24 is a conduit through which a liquid such as saline or saline solution flowing out from a priming syringe interlocked to the port 26 flows.

A constituent material from which the tube 24 may be fabricated is not particularly limited as long as the constituent material is a flexible resin material in consideration of operability. As the constituent material for the tube 24, polyolefins such as polyethylene, polypropylene, and ethylene-propylene copolymer, polyester such as polyethylene terephthalate, polystyrene, or polyvinyl chloride can be suitably used.

The three-way stopcock 25 communicates with the loading lumen 22 of the catheter body 21 via the insertion passage 23a of the proximal hub 23 and the tube 24. In addition to a proximal side of the tube 24, a priming syringe for priming the loading lumen 22 of the catheter body 21 can be connected to the port 26 of the three-way stopcock 25.

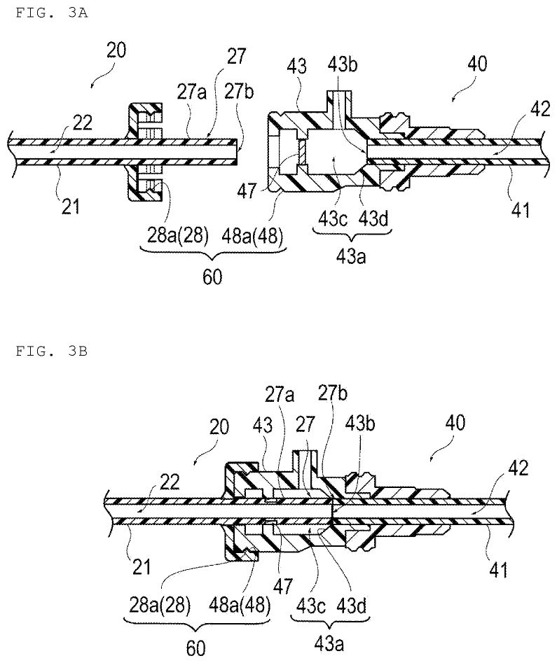

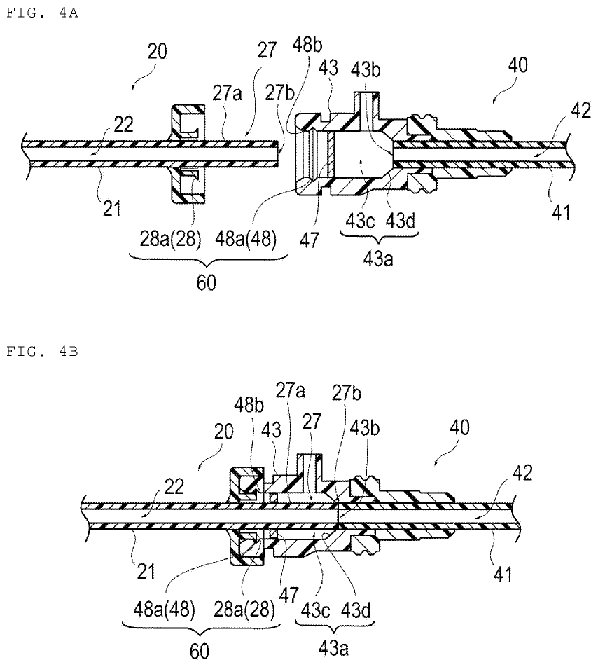

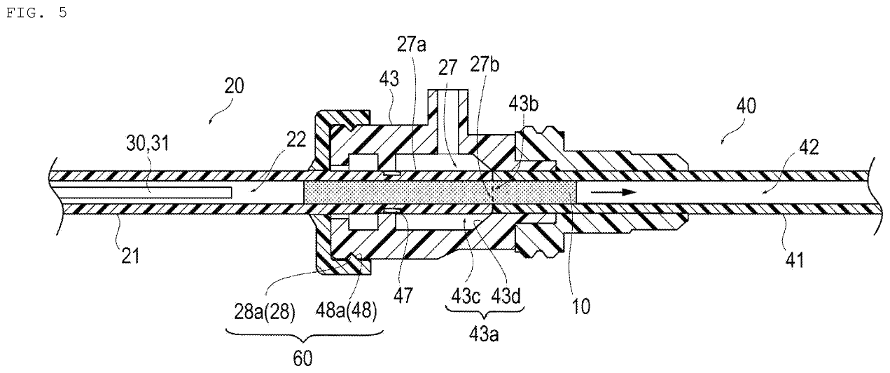

As shown in FIGS. 3 and 4, a distal side of the embolic device loading catheter 20 is provided with the distal connection portion 27 that is connected to the sheath hub 43 of the delivery catheter 40. The distal connection portion 27 is provided with a second engagement portion 28 that engages with a first engagement portion 48 provided on a proximal side of the sheath hub 43. In the medical instrument set 100 according to the present embodiment, the first engagement portion 48 and the second engagement portion 28 constitute the engagement portion 60 for maintaining a connected state between the embolic device loading catheter 20 and the delivery catheter 40.

FIGS. 3 and 4 show configuration examples of the engagement portion 60. In the engagement portion 60 of each configuration, the first engagement portion 48 functions as a female portion, and the second engagement portion 28 functions as a male portion.

As shown in FIG. 3A, the engagement portion 60 may be configured such that the embolic device loading catheter 20 is fitted outside the delivery catheter 40. In this configuration, the first engagement portion 48 is provided as a groove portion 48a provided along a circumferential direction on an outer circumferential surface of the sheath hub 43 on the proximal side. The second engagement portion 28 is provided as a separate collar member (collar) fixed on an outer circumferential surface of a distal portion of the catheter body 21, and includes a plurality of engagement claws 28a provided so as to cover at least a part of the outer circumference of the distal portion of the catheter body 21. As shown in FIG. 3B, the engagement portion 60 maintains the connected state between the embolic device loading catheter 20 and the delivery catheter 40 by the engagement claws 28a of the second engagement portion 28 fitted into the groove portion 48a, which is the first engagement portion 48. When the embolic device loading catheter 20 and the delivery catheter 40 are in the connected state, the loading lumen 22 and the sheath lumen 42 communicate with each other.

As shown in FIG. 4A, the engagement portion 60 may be configured such that the embolic device loading catheter 20 is fitted inside the delivery catheter 40. In this configuration, the first engagement portion 48 is provided as a groove portion 48a formed in an inner wall surface of an inserting portion 48b, which is a passage provided in a substantially central portion of an end surface of the sheath hub 43 on the proximal side. The second engagement portion 28 is provided as a separate skirt member fixed on the outer circumferential surface of the distal portion of the catheter body 21, and includes a plurality of engagement claws 28a disposed so as to be able to fit into the groove portion 48a when the second engagement portion 28 protrudes in a distal direction and is inserted into the inserting portion 48b. As shown in FIG. 4B, the engagement portion 60 maintains the connected state between the embolic device loading catheter 20 and the delivery catheter 40 by the engagement claws 28a fitted into the groove portion 48a. When the embolic device loading catheter 20 and the delivery catheter 40 are in the connected state, the loading lumen 22 and the sheath lumen 42 communicate with each other.

A configuration of the engagement portion 60 is not limited to fitting forms shown in FIGS. 3 and 4 as long as the connected state between the embolic device loading catheter 20 and the delivery catheter 40 is maintained, and can also employ other connection configurations, for example, such as a threaded type. The engagement portion 60 is configured to maintain a connected state between the embolic device loading catheter 20 and the delivery catheter 40, and prevents the connected state of the embolic device loading catheter 20 and the delivery catheter 40 from being detached during the procedure. However, the embolic device loading catheter 20 and the delivery catheter 40 do not necessarily have to be engaged via the engagement portion 60. For example, a state where the distal connection portion 27 is inserted into the sheath hub 43 may be the connected state between the embolic device loading catheter 20 and the delivery catheter 40.

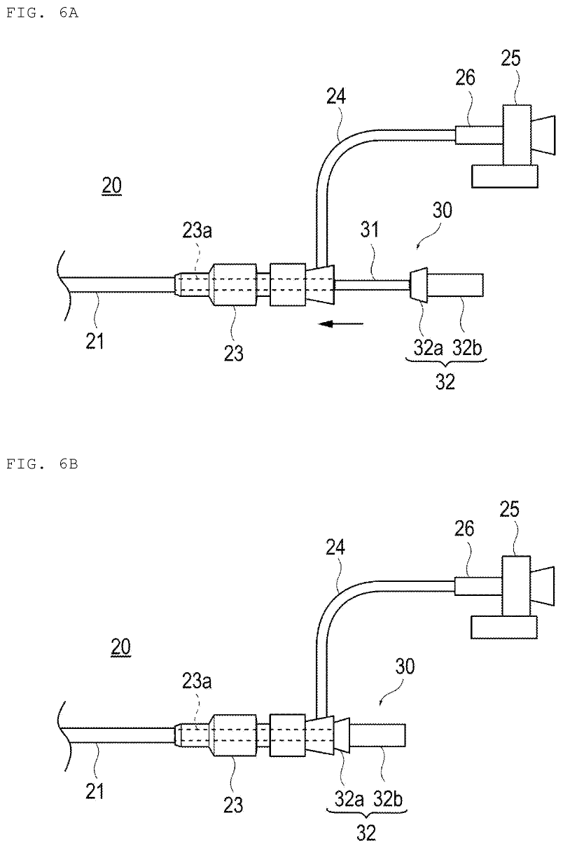

As shown in FIG. 5, the inner diameter of the loading lumen 22 is designed to be substantially equal to the inner diameter of the sheath lumen 42, and accordingly, the outer diameter of the embolic device 10 is also designed, and the embolic device 10 can have a diameter size larger than that of the direct insertion method or the indirect insertion method. Accordingly, in the endoleak embolization, the number of inserted embolic device 10 can be reduced, and as a result, the procedure time can be shortened. Since the inner diameter of the loading lumen 22 is substantially equal to the inner diameter of the sheath lumen 42, in the connected state between the embolic device loading catheter 20 and the delivery catheter 40 caused by the engagement portion 60, a step (clearance) between an opening portion of a distal contact portion 27b and an opening portion on a proximal side of a communication distal portion 43b can be made close to zero. Therefore, when a pusher body 31 of the delivery pusher 30 is inserted into the sheath lumen 42 from the loading lumen 22, the embolic device 10 can be smoothly moved from the embolic device loading catheter 20 to the delivery catheter 40.

The distal connection portion 27 includes an insertion portion 27a that is inserted into a communicating enlarged-diameter portion 43c provided inside the sheath hub 43 when the embolic device loading catheter 20 and the delivery catheter 40 are in the connected state. In the connected state between the embolic device loading catheter 20 and the delivery catheter 40, the insertion portion 27a is inserted into the sheath hub 43 such that the loading lumen 22 and the sheath lumen 42 are aligned in the axial direction. Accordingly, the embolic device 10 is pushed out to the sheath lumen 42 without being exposed to an outside from the loading lumen 22 via the sheath hub 43.

In the connected state between the embolic device loading catheter 20 and the delivery catheter 40, the insertion portion 27a includes the distal contact portion 27b, on a distal side of the insertion portion 27a, that comes into contact with an inner surface of a tapered portion 43d provided in the communicating enlarged-diameter portion 43c. When the insertion portion 27a is inserted into the communicating enlarged-diameter portion 43c, the distal contact portion 27b comes into contact with the tapered portion 43d, so that the loading lumen 22 and the sheath lumen 42 communicate with each other so as not to intersect with other spaces including a space of the communicating enlarged-diameter portion 43c. Therefore, when the embolic device 10 is pushed out from the embolic device loading catheter 20 into the delivery catheter 40, the embolic device 10 can be prevented from being damaged (for example, bent or crushed on a distal side) due to abutment of the embolic device 10 against an inner wall surface of the sheath hub 43, can be prevented from being erroneously inserted into other spaces within the sheath hub 43 (for example, erroneously entering into a tube 44 connected to the sheath hub 43), and can be reliably injected into the sheath lumen 42.

Delivery Pusher

The delivery pusher 30 is an elongated rod-shaped member inserted into the proximal hub 23 and configured to push out the embolic device 10 accommodated in the catheter body 21 and deliver the embolic device 10 into the aneurysm via the sheath lumen 42 of the delivery catheter 40. The delivery pusher 30 can include the rod-shaped pusher body 31 and a handle portion 32 provided on a proximal side of the pusher body 31 and held by the surgeon when the embolic device 10 is to be delivered into the aneurysm.

In a state where the embolic device loading catheter 20 is connected to the delivery catheter 40, when the surgeon performs a predetermined operation while gripping the handle portion 32, the delivery pusher 30 pushes the embolic device 10 loaded in the loading lumen 22 into the aneurysm via the sheath lumen 42 of the delivery catheter 40. Specifically, the delivery pusher 30 is pushed out along axial directions of the embolic device loading catheter 20 and the delivery catheter 40 to push out the embolic device 10 loaded in the embolic device loading catheter 20 to an outside (into the aneurysm).

In the connected state where the embolic device loading catheter 20 is connected to the delivery catheter 40, a body length of the pusher body 31 of the delivery pusher 30 is longer than a distance from a proximal end of the insertion passage 23a of the proximal hub 23 to the distal end opening 41a of the sheath 41 of the delivery catheter 40 (an opening portion on a distal side communicating with the sheath lumen 42). Therefore, in the state where the embolic device loading catheter 20 is connected to the delivery catheter 40, when the delivery pusher 30 is inserted from the proximal hub 23, the embolic device 10 loaded in the loading lumen 22 can be passed through the insertion passage 23a into the sheath hub 43, and then the embolic device 10 can be passed into the sheath lumen 42 and pushed out into the aneurysm by a single push-out operation.

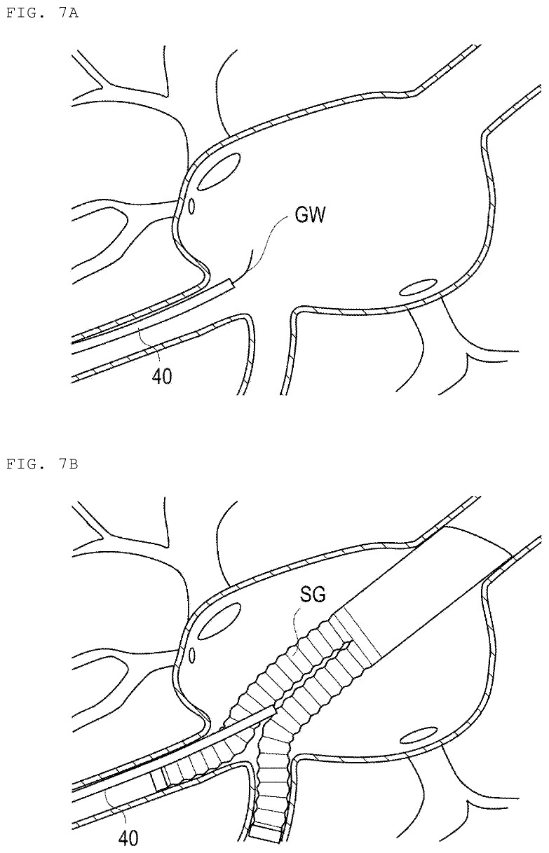

As shown in FIG. 6A, the handle portion 32 has a substantially mushroom shape having a large-diameter head portion 32a on a distal side and a small-diameter handle portion 32b extending to a proximal side of the large-diameter head portion 32a, and an outer diameter dimension of the large-diameter head portion 32a, which is a maximum outer diameter of the handle portion 32, is designed to be larger than an inner diameter dimension of the insertion passage 23a of the proximal hub 23. Accordingly, as shown in FIG. 6B, when the delivery pusher 30 is inserted into the embolic device loading catheter 20, since the large-diameter head portion 32a is not inserted into the insertion passage 23a of the proximal hub 23, an insertion length of the delivery pusher 30 can be limited. Since the handle portion 32 does not enter the proximal hub 23 due to the large-diameter head portion 32a, when the push-out operation of the embolic device 10 by the delivery pusher 30 is completed, the handle portion 32 can be rather easily pulled out simultaneously in the inserted state in accordance with a detachment operation of the embolic device loading catheter 20, thereby simplifying a detachment operation. The delivery pusher 30 may be pulled out from the embolic device loading catheter 20 before the detachment operation of the embolic device loading catheter 20.

The handle portion 32 may be configured such that the large-diameter head portion 32a can be fitted to the proximal side of the proximal hub 23 when the handle portion 32 is inserted into the embolic device loading catheter 20. With such a configuration, when the embolic device loading catheter 20 is detached, the delivery pusher 30 can be rather reliably pulled out without being detached from the embolic device loading catheter 20.

A constituent material from which the pusher body 31 may be fabricated is not particularly limited as long as the constituent material is a material having appropriate hardness and flexibility such that the embolic device 10 can be conveyed. As examples of the constituent material for the pusher body 31, a resin material such as a polymer material including polyolefins (for example, polyethylene, polypropylene, polybutene, an ethylene-propylene copolymer, an ethylene-vinyl acetate copolymer, an ionomer, or a mixture of two or more types thereof), a polyolefin elastomer, a crosslinked polyolefin, polyvinyl chloride, polyamide, a polyamide elastomer, polyester, polyester elastomer, polyurethane, polyurethane elastomer, a fluorous resin such as ETFE, polycarbonate, polystyrene, polyacetal, polyimide, polyetherimide, and aromatic polyether ketone, or a mixture thereof, and a metal material such as a shape memory alloy, stainless steel, tantalum, titanium, platinum, gold, or tungsten can be suitably used.

Delivery System

Next, the delivery system 200 according to the present embodiment will be described. As shown in FIG. 1, the delivery system 200 according to the present embodiment can include, in addition to the medical instrument set 100, the delivery catheter 40 to and from which the embolic device loading catheter 20 is attached and detached while indwelling in the body lumen.

The delivery catheter 40 can also use, for example, an existing catheter that can indwell in the body lumen. Therefore, in the delivery system 200 according to the present embodiment, the medical instrument set 100 and the delivery catheter 40 can be sold as a set and supplied to a market, but even when only the medical instrument set 100 is sold and supplied to the market, an existing catheter can be used as the delivery catheter 40 to function as the delivery system 200.

Delivery Catheter

The delivery catheter 40 can include, for example, the sheath 41 formed of an elongated tubular member in which a hole (the sheath lumen 42) extending from an opening portion on a distal side of the delivery catheter 40 to an opening portion on a proximal side of the delivery catheter 40 along an axial direction is formed, and indwells in the body lumen and functions as an introduction passage for delivering the embolic device 10 into the aneurysm. A main body 51 of an insertion assisting member 50, which will be described later, can be inserted through the sheath 41 over a total length of the sheath 41. Therefore, a length of the sheath 41 in the axial direction is set to be at least shorter than a length of the main body 51 of the insertion assisting member 50.

As shown in FIG. 5, the inner diameter of the sheath lumen 42 is designed to be substantially equal to the inner diameter of the loading lumen 22. Accordingly, the embolic device 10 can be relatively smoothly moved from the loading lumen 22 to the sheath lumen 42 when the embolic device loading catheter 20 and the delivery catheter 40 are in the connected state by the engagement portion 60.

A constituent material from which the sheath 41 may be fabricated is not particularly limited as long as the constituent material is flexible and rigid enough to follow the bent shape of the body lumen such as meandering and bending. As examples of the constituent material for the sheath 41, a resin material such as a polymer material including polyolefins (for example, polyethylene, polypropylene, polybutene, an ethylene-propylene copolymer, an ethylene-vinyl acetate copolymer, an ionomer, or a mixture of two or more types thereof), a polyolefin elastomer, a crosslinked polyolefin, polyvinyl chloride, polyamide, a polyamide elastomer, polyester, polyester elastomer, polyurethane, polyurethane elastomer, a fluorous resin, polycarbonate, polystyrene, polyacetal, polyimide, polyetherimide, and aromatic polyether ketone, or a mixture thereof can be suitably used.

The delivery catheter 40 includes the sheath hub 43 interlocked to the proximal side of the sheath 41, and the flexible tube 44 having one end connected to the proximal side of the sheath hub 43 and the other end connected to a three-way stopcock 45.

The sheath hub 43 is an intermediate member that includes a communication passage 43a that allows communication between the sheath lumen 42 and the tube 44 and between the loading lumen 22 and the sheath lumen 42, and allows a fluid (such as saline (or saline solution) for priming) flowing from the three-way stopcock 45 to flow to the sheath 41 via the tube 44 and to guide the embolic device 10 pushed out from the embolic device loading catheter 20 into the sheath lumen 42. The insertion assisting member 50 is inserted into the sheath hub 43 when the delivery catheter 40 indwells in the body lumen.

A constituent material from which the sheath hub 43 may be fabricated may be the same as the material exemplified as the constituent material for the proximal hub 23 described above.

The communication passage 43a includes the communication distal portion 43b having an inner diameter substantially equal to the inner diameter of the sheath lumen 42, and the communicating enlarged-diameter portion 43c extending from the communication distal portion 43b in a proximal direction and serving as an internal space having a diameter larger than that of the communication distal portion 43b. The tapered portion 43d increased in diameter in the proximal direction from an opening portion on the proximal side of the communication distal portion 43b is provided in a portion of the communicating enlarged-diameter portion 43c connected to the communication distal portion 43b.

In the connected state between the embolic device loading catheter 20 and the delivery catheter 40, a distal end of the loading lumen 22 is inserted so as to be adjacent to the opening portion on the proximal side of the communication distal portion 43b. Accordingly, when the embolic device 10 is pushed out from the embolic device loading catheter 20 into the delivery catheter 40, the embolic device 10 can be emitted from the loading lumen 22 into the sheath lumen 42 without being exposed to the outside.

The insertion state of the insertion portion 27a is preferably such that the inner surface of the tapered portion 43d and the distal contact portion 27b come into contact with each other so that the loading lumen 22 and the sheath lumen 42 are aligned in the axial direction. Accordingly, since a distal end of the insertion portion 27a and the opening portion on the proximal side of the communication distal portion 43b are connected along the axial direction, the pushed-out embolic device 10 can be prevented from being damaged (for example, be bent or crushed on the distal side) due to abutment of the embolic device 10 against the inner wall surface of the sheath hub 43, and the loading lumen 22 and the sheath lumen 42 are communicated with each other so as not to intersect with a space inside the communicating enlarged-diameter portion 43c, thereby preventing the embolic device 10 from being erroneously inserted into other spaces within the sheath hub 43 (for example, erroneously entering into the tube 44 connected to the sheath hub 43).

As shown in FIGS. 3 and 4, the proximal side of the sheath hub 43 is provided with the first engagement portion 48 to be engaged with the second engagement portion 28 provided at the distal connection portion 27 of the embolic device loading catheter 20.

For example, the first engagement portion 48 may be configured by the groove portion 48a into which the engagement claws 28a of the second engagement portion 28 described above are fitted. The groove portion 48a is provided along the circumferential direction on the outer circumferential surface of the sheath hub 43 on the proximal side in FIG. 3, and is provided along a circumferential direction on the inner wall surface of the inserting portion 48b formed in a substantially central portion of the end surface of the sheath hub 43 on the proximal side in FIG. 4.

The one end of the tube 44 is interlocked to the proximal side of the sheath hub 43, and the other end is interlocked to the port 46 of the three-way stopcock 45. The tube 44 is a conduit through which a liquid such as a saline (or saline solution) flows from a priming syringe interlocked to the port 46. A constituent material from which the tube 44 may be fabricated may be the same as the material exemplified as the constituent material for the tube 24 described above.

The three-way stopcock 45 communicates with the sheath lumen 42 of the sheath 41 via the communication passage 43a of the sheath hub 43 and the tube 44. In addition to a proximal side of the tube 44, a priming syringe for priming the sheath lumen 42 of the sheath 41 and a liquid agent injection syringe for injecting a contrast agent, a drug, or the like can be connected to the port 46 of the three-way stopcock 45.

A hemostasis valve 47 can be attached into the proximal side of the sheath hub 43. As the hemostasis valve 47, for example, a substantially elliptical film-shaped (disk-shaped) valve body made of silicone rubber, latex rubber, butyl rubber, or isoprene rubber which is an elastic member may be used. When the embolic device loading catheter 20 is connected to the delivery catheter 40, at least the distal contact portion 27b of the insertion portion 27a passes through the hemostasis valve 47 and is inserted into the communicating enlarged-diameter portion 43c.

Here, an example of dimensions of each device constituting the delivery system 200 according to the present embodiment will be described. Numerical values shown below are merely examples, and the present disclosure is not limited thereto.

In the delivery system 200 according to the present embodiment, when the delivery catheter 40 is a catheter having an outer diameter of 6 Fr (an inner diameter of 1.8 mm) and a surgical method to be applied is endoleak embolization for stent graft interpolation of an abdominal aortic aneurysm (AAA), the outer diameter of the embolic device 10 can be, for example, 0.4 mm to 1.9 mm (preferably about 1.6 mm), and the inner diameter of the embolic device loading catheter 20 can be, for example, 1.0 mm to 1.8 mm (preferably about 1.8 mm), which is equal to the inner diameter of the delivery catheter 40. A body length of the catheter body 21 of the embolic device loading catheter 20 can be, for example, 30 cm to 105 cm (preferably about 42 cm), a body length of the sheath 41 of the delivery catheter 40 can be, for example, 39 cm to 90 cm (preferably about 47 cm), and a body length of the pusher body 31 of the delivery pusher 30 can be, for example, 79 cm to 205 cm (preferably about 96 cm). The total length of the embolic device 10 is appropriately determined depending on a size of the aneurysm, and may be in a range, for example, of 30 cm to 100 cm (preferably about 40 cm) from the viewpoint of ease of loading into the embolic device loading catheter 20 and shortening the procedure time.

Embolic Device Delivery Medical System

Next, the embolic device delivery medical system 300 according to the present embodiment will be described. As shown in FIG. 2, the embolic device delivery medical system 300 according to the present embodiment can include, in addition to the delivery system 200, the insertion assisting member 50 that delivers the delivery catheter 40 into the body lumen.

Insertion Assisting Member

The insertion assisting member 50 is an auxiliary tool that is provided with a guide wire lumen 52 that is inserted from a distal side to a proximal side along an axial direction of the main body 51, and assists insertion when the delivery catheter 40 is delivered into the aneurysm along a guide wire inserted in advance into the body lumen.

The insertion assisting member 50 is inserted into and assembled to the delivery catheter 40 in order to help prevent bending or the like when the delivery catheter 40 is inserted into the body lumen. The guide wire lumen 52 has an inner diameter smaller than that of the sheath lumen 42 of the delivery catheter 40. Therefore, when the delivery catheter 40 is delivered into the aneurysm, axial deviation of the delivery catheter 40 with respect to the guide wire can be reduced, which makes delivery relatively easier.

A constituent material from which the insertion assisting member 50 may be fabricated is not particularly limited as long as the constituent material is harder and more flexible than a constituent material for the delivery catheter 40. As an example of the constituent material for the insertion assisting member 50, polyolefins such as polyethylene or polypropylene, polyester such as polyamide or polyethylene terephthalate, a fluorous resin such as ETFE, a resin material such as PEEK (polyether ether ketone) or polyimide, a metal material such as a shape memory alloy, stainless steel, tantalum, titanium, platinum, gold, or tungsten can be suitably used.

Operations

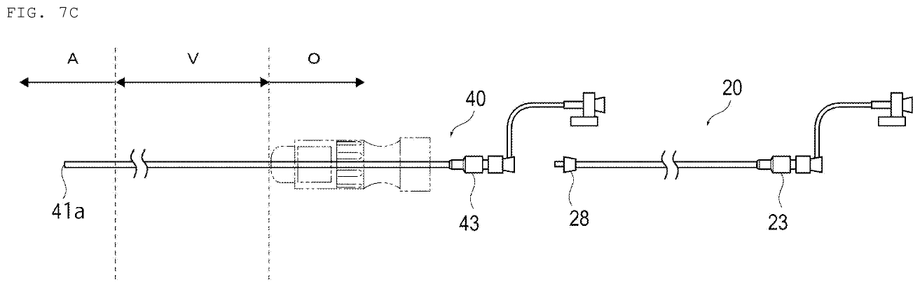

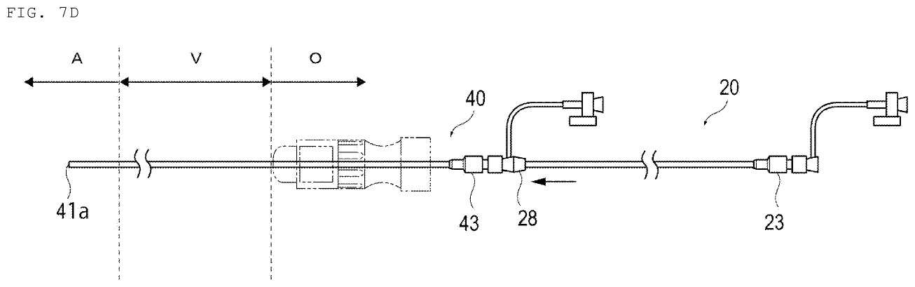

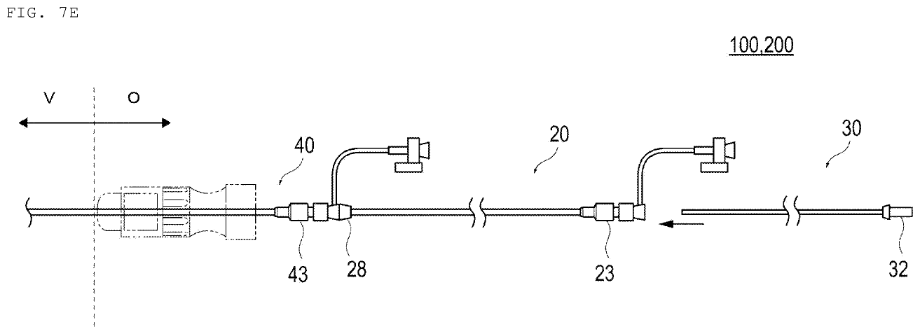

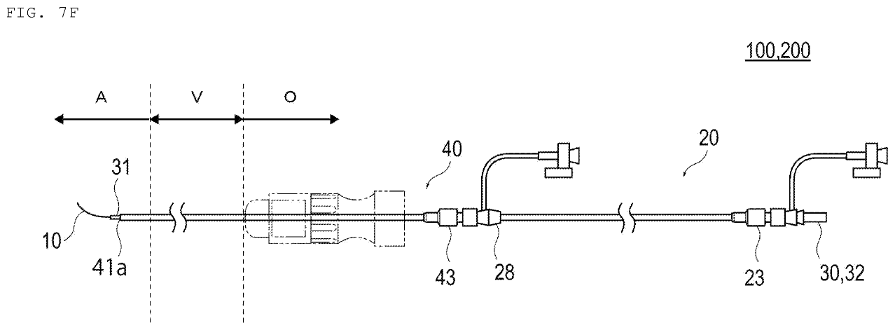

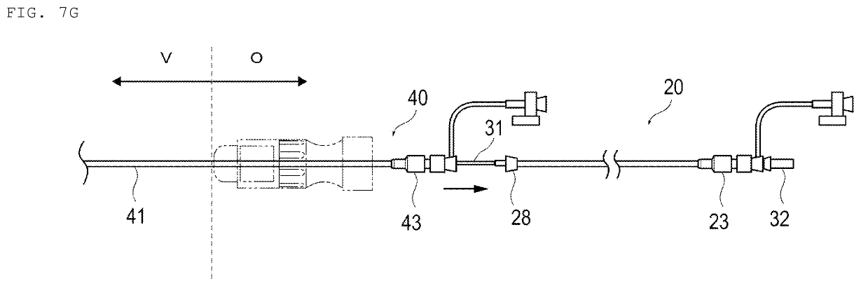

Next, an operation of the embolic device delivery medical system 300 according to the present embodiment will be described with reference to FIGS. 7A to 7G. Here, an operation example when the embolic device delivery medical system 300 is applied to the endoleak embolization for the stent graft interpolation of the abdominal aortic aneurysm (AAA) is described, and configuration examples of the engagement portion 60 are shown in FIGS. 3A and 3B. In each drawing, the inside of the aneurysm is represented by “A”, the inside of the blood vessel is represented by “V”, the outside of the body is represented by “0”, and installation positions of the devices of the embolic device delivery medical system 300 are expressed so as to be systematically grasped.

First, as a preparation step before surgery, as shown in FIG. 7A, the surgeon percutaneously inserts, from a limb of a patient serving as a puncture site into the body lumen via an introducer (for example, a member indicated by a two-dot chain line in FIG. 7A), the sheath 41 of the delivery catheter 40 into which the insertion assisting member 50 is inserted, and delivers a distal end opening 41a of the delivery catheter 40 to the abdominal aortic aneurysm. When the distal end opening 41a is delivered into the aneurysm, the insertion assisting member 50 is removed. The delivery catheter 40 may be delivered to an aneurysm lesion by using the guide wire inserted in the aneurysm in advance without using the insertion assisting member 50.

Next, as shown in FIG. 7B, the surgeon inserts, into the body lumen via the introducer, a catheter (a stent graft device) into which a stent graft SG is compressed and inserted, and moves the catheter to the aneurysm lesion using the guide wire inserted in advance into the aneurysm. Thereafter, the stent graft SG is expanded from the catheter and indwells in the target lesion. Accordingly, as shown in FIG. 7B, the delivery catheter 40 can be inserted between a leg of the stent graft SG and a blood vessel wall, a distal portion of the delivery catheter 40 can be inserted between the stent graft SG and a blood vessel wall of the aneurysm, that is, into the aneurysm, and the delivery catheter 40 indwells in the body lumen with the distal end opening 41a positioned in the aneurysm.

As shown in FIG. 7C, the embolic device loading catheter 20 loaded with the embolic device 10 is prepared. FIG. 7C shows a state before the embolic device loading catheter 20 is connected to the delivery catheter 40.

When the delivery catheter 40 indwells, the surgeon connects the distal connection portion 27 of the embolic device loading catheter 20 to a proximal end of the sheath hub 43 of the delivery catheter 40, as shown in FIG. 7D. At this time, as shown in FIG. 3B, the distal contact portion 27b of the distal connection portion 27 is inserted into the communicating enlarged-diameter portion 43c of the sheath hub 43 and comes into contact with the inner surface of the tapered portion 43d. Accordingly, the distal end of the loading lumen 22 can be adjacent to the communication distal portion 43b, and an axis of the loading lumen 22 and an axis of the sheath lumen 42 can be aligned.

Next, the surgeon inserts a distal end of the pusher body 31 from the proximal side of the proximal hub 23 while gripping the handle portion 32, as shown in FIG. 7E. A distal end of the delivery pusher 30 inserted from the proximal hub 23 comes into contact with a proximal end of the embolic device 10 loaded in the embolic device loading catheter 20, and the embolic device 10 is pushed out and moved to the sheath lumen 42 of the delivery catheter 40 by the push-out operation.

As shown in FIG. 7F, the surgeon pushes out the delivery pusher 30 inserted from the proximal hub 23 to push out the embolic device 10 from the sheath lumen 42 into the aneurysm. Thereafter, the surgeon detaches the emptied embolic device loading catheter 20 together with the delivery pusher 30 from the delivery catheter 40, as shown in FIG. 7G. The delivery pusher 30 can be detached from the delivery catheter 40 while being inserted into the embolic device loading catheter 20. Thus, a first insertion operation of the embolic device 10 into the aneurysm is completed. In the insertion operation, the delivery pusher 30 may be pulled out from the embolic device loading catheter 20 before the detachment operation of the embolic device loading catheter 20. Such a series of embolic device indwelling operations shown in FIGS. 7C to 7G are repeated until a required amount (or desired amount) of embolic device 10 is loaded into the aneurysm. The required amount (or desired amount) of the embolic device 10 can be calculated as a value obtained by calculating a volume of the aneurysm based on CT data about the patient and subtracting the volume of the stent graft SG when the stent graft SG is expanded in the aneurysm from the calculated value.

After the required amount of embolic device 10 has indwelled in the aneurysm, the surgeon pulls out the delivery catheter 40 from the aneurysm and the body lumen. At this time, the delivery catheter 40 may be pulled out from the aneurysm and the body lumen in a state where the embolic device loading catheter 20 is connected to the delivery catheter 40 and the delivery pusher 30 is inserted into the delivery catheter 40. Before the delivery catheter 40 is pulled out from the aneurysm and the body lumen, the delivery pusher 30 may be pulled out from the delivery catheter 40 while the embolic device loading catheter 20 is detached from the delivery catheter 40. Before the delivery catheter 40 is pulled out from the aneurysm and the body lumen, the delivery pusher 30 may be pulled out from the delivery catheter 40 and the embolic device loading catheter 20, and then the embolic device loading catheter 20 may be detached from the delivery catheter 40. In any case, the introducer remains indwelled in the body lumen for additional expansion of the stent graft SG by a balloon after the embolic device 10 indwells, an imaging operation, and the like.

Thereafter, the embolic device 10 indwelling in the aneurysm comes into contact with a liquid such as blood in the aneurysm and gradually swells, and the completely expanded embolic device 10 fills a space between an inner surface of the aneurysm and an outer surface of the stent graft, thereby closing the aneurysm. Accordingly, the aneurysm is prevented from rupture.

As described above, the embolic device delivery medical system 300 including the medical instrument set 100 and the delivery system 200 according to the present embodiment can deliver, for example, the embolic device 10 into the aneurysm only in three procedures as shown in FIGS. 7A to 7C. Therefore, the number of procedure steps can be reduced, and the procedure time can be shortened as compared with the indirect insertion method which can be assumed as the delivery method of the embolic device 10.

Modification

The embodiment described above can also be modified to have the following configuration.

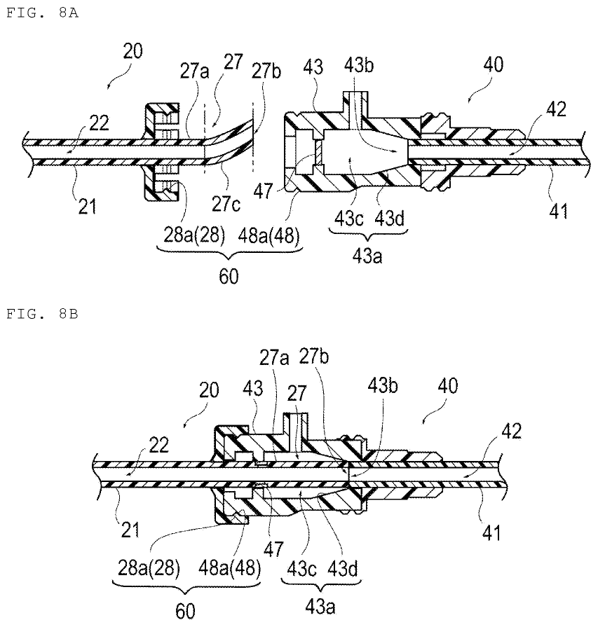

FIG. 8 shows another configuration example of the insertion portion 27a of the distal connection portion 27 in the embolic device loading catheter 20. As shown in FIG. 8A, the insertion portion 27a according to a modification includes a bending portion 27c. The bending portion 27c forms at least a part of the insertion portion 27a, and has a shape that is gradually bent in a direction away from a central axis of the loading lumen 22 (a radial direction with respect to the central axis of the loading lumen 22) toward a distal side of the loading lumen 22 (the distal contact portion 27b). The bending portion 27c may be provided, for example, in a region partitioned by a two-dot chain line shown in FIG. 8A in the insertion portion 27a of the embolic device loading catheter 20. In the shown example, a part of an intermediate portion located between a proximal end of the insertion portion 27a and the distal contact portion 27b (an opening portion of the distal end) is formed by the bending portion 27c, and is bent in a bent shape.

As shown in FIG. 8B, when the bending portion 27c is inserted into the communicating enlarged-diameter portion 43c of the sheath hub 43, the bent shape of the bending portion 27c is corrected into a substantially straight state. In the connected state where the embolic device loading catheter 20 is connected to the delivery catheter 40, the bending portion 27c is corrected such that the axis of the loading lumen 22 and the axis of the sheath lumen 42 of the delivery catheter 40 are aligned in a state where the distal contact portion 27b is in contact with the inner surface of the tapered portion 43d of the sheath hub 43. That is, when the bending portion 27c is inserted into the communicating enlarged-diameter portion 43c, the bending portion 27c comes into contact with an inner wall or the like of the communicating enlarged-diameter portion 43c, whereby the bent shape of the bending portion 27c is corrected into a substantially straight shape. In particular, the distal contact portion 27b comes into contact with the inner wall of the communicating enlarged-diameter portion 43c, and a proximal portion of the insertion portion 27a comes into contact with the hemostasis valve 47 of the sheath hub or the inner wall of the opening portion on a proximal side of the sheath hub, whereby the bending portion 27c is corrected into a substantially straight shape.

The bending portion 27c has a bent shape with a predetermined curvature smoothly deformed in an arch shape from the proximal end of the insertion portion 27a to the distal contact portion 27b (an opening portion of the distal end) or from the intermediate portion of the insertion portion 27a to the distal contact portion 27b (an opening portion of the distal end), and also has a bent shape such as a substantially bent shape bent from a certain starting point.

A constituent material from which the bending portion 27c may be fabricated is not particularly limited as long as the constituent material has flexibility such that the bending portion 27c keeps a bent state before being inserted into the sheath hub 43, comes into contact with the inner wall or the like of the communicating enlarged-diameter portion 43c and can be elastically deformed into a substantially straight state after being inserted into the sheath hub 43. Since the bending portion 27c only needs to have lower rigidity than the sheath hub 43, in order to produce a difference in rigidity, the bending portion 27c may be structured by changing the constituent material, or by changing a thickness of the bending portion 27c when the same material is employed.

Thus, by providing the bending portion 27c in at least a part of the insertion portion 27a, it is possible to prevent the embolic device 10 loaded in the loading lumen 22 from erroneously falling off from a distal end of the distal connection portion 27. When the bending portion 27c is inserted into the sheath hub 43, the bent shape of the bending portion 27c is corrected to a substantially straight state, so that the axis of the loading lumen 22 and the axis of the sheath lumen 42 are aligned.

Therefore, even if the insertion portion 27a is partially bent, the insertion of the embolic device 10 is not hindered.

Functions and Effects

As described above, the medical instrument set 100 according to the present embodiment is configured to deliver the embolic device 10 into an aneurysm via the delivery catheter 40. The delivery catheter 40 includes the sheath 41 that includes the sheath lumen 42 and the distal end opening 41a that communicates with the sheath lumen 42, and the sheath hub 43 that includes the communication passage 43a communicating with a proximal end of the sheath lumen 42 and is provided on the proximal side of the sheath 41. The medical instrument set 100 includes: the embolic device loading catheter 20 that includes the catheter body 21 including the loading lumen 22 having an open distal end, and the proximal hub 23 including the insertion passage 23a communicating with a proximal end of the loading lumen 22, and in which the embolic device 10 is loaded in the loading lumen 22; and the delivery pusher 30 that includes the elongated pusher body 31 insertable into the loading lumen 22 through the insertion passage 23a of the proximal hub 23 of the embolic device loading catheter 20. The catheter body 21 includes the distal connection portion 27 attachable to and detachable from the sheath hub 43 of the delivery catheter 40 on the distal side of the catheter body 21. The pusher body 31 has a body length longer than a distance from the proximal end of the insertion passage 23a of the proximal hub 23 to the distal end opening 41a of the sheath 41 of the delivery catheter 40 in the connected state where the distal connection portion 27 of the catheter body 21 is connected to the sheath hub 43 of the delivery catheter 40, and when the pusher body 31 is inserted from the insertion passage 23a of the proximal hub 23 in the connected state, the embolic device 10 is passed through the sheath lumen 42 of the delivery catheter 40 and is pushed out from the distal end opening 41a of the delivery catheter 40 into the aneurysm.

The pusher body 31 of the delivery pusher 30 is formed so as to be longer than the distance from the proximal end of the insertion passage 23a of the proximal hub 23 to the distal end opening 41a of the sheath 41 of the delivery catheter 40 in the connected state between the embolic device loading catheter 20 and the delivery catheter 40. Therefore, in the connected state where the embolic device loading catheter 20 is connected to the delivery catheter 40, by inserting the delivery pusher 30 from the proximal hub 23, the embolic device 10 loaded in the loading lumen 22 can be pushed out into the aneurysm by a single push-out operation. Therefore, the number of procedure steps can be reduced. In addition, since it is only necessary to connect the embolic device loading catheter 20 to the sheath hub 43 of the delivery catheter 40, the procedure can be simplified, and as a result, the procedure time can be shortened.

In the medical instrument set 100 according to the present embodiment, preferably, the inner diameter of the loading lumen 22 may be substantially equal to the inner diameter of the sheath lumen 42.

With such a configuration, the outer diameter of the embolic device 10 loaded into the loading lumen 22 can be made substantially equal to the inner diameters of the loading lumen 22 and the sheath lumen 42. Therefore, the diameter size of the embolic device 10 can be made large without being made small in accordance with the loading lumen 22 as in the direct insertion method and the indirect insertion method, so that the number of inserted embolic device 10 can be reduced and the procedure time can be shortened.

In the medical instrument set 100 according to the present embodiment, preferably, the distal connection portion 27 of the catheter body 21 may include the insertion portion 27a that is inserted into the sheath hub 43 of the delivery catheter 40 in the connected state.

With such a configuration, when the embolic device loading catheter 20 is connected to the delivery catheter 40, the insertion portion 27a can be inserted into the sheath hub 43, so that the embolic device 10 can be pushed out from the loading lumen 22 to the sheath lumen 42 via the sheath hub 43 without being exposed to the outside.

In the medical instrument set 100 according to the present embodiment, preferably, the delivery pusher 30 may include the handle portion 32 provided on the proximal side of the pusher body 31, and the maximum outer diameter of the handle portion 32 may be larger than an inner diameter of the insertion passage 23a of the proximal hub 23.

With such a configuration, when the delivery pusher 30 is inserted into the embolic device loading catheter 20, since a distal end of the handle portion 32 (the large-diameter head portion 32a) is not inserted into the insertion passage 23a of the proximal hub 23, an insertion length of an insertion pusher of the delivery pusher 30 can be limited. Since the handle portion 32 does not enter the proximal hub 23, when the push-out operation of the embolic device 10 by the delivery pusher 30 is completed, the delivery pusher 30 can be rather easily pulled out simultaneously with the detachment operation of the embolic device loading catheter 20.

The delivery system 200 according to the present embodiment includes the medical instrument set 100 and the delivery catheter 40, and is configured such that, in the connected state, the embolic device 10 is passed through the sheath lumen 42 of the delivery catheter 40 by the delivery pusher 30 inserted from the insertion passage 23a of the proximal hub 23 and is pushed out from the distal end opening 41a of the delivery catheter 40 into the aneurysm.

When the distal connection portion 27 of the embolic device loading catheter 20 is engaged with the sheath hub 43 of the delivery catheter 40 indwelling in the body lumen to be in the connected state, the loading lumen 22 and the sheath lumen 42 are in communication with each other through the communication passage 43a of the sheath hub 43. Therefore, by inserting the delivery pusher 30 from the proximal hub 23, the embolic device 10 loaded in the loading lumen 22 can be pushed out into the aneurysm by a single push-out operation.

The delivery system 200 according to the present embodiment may be configured such that, preferably, the delivery catheter 40 includes the first engagement portion 48 including the groove portion 48a provided on the proximal side of the sheath hub 43, the embolic device loading catheter 20 includes the second engagement portion 28 including the engagement claws 28a to be engaged with the first engagement portion 48 in the distal connection portion 27, and the first engagement portion 48 and the second engagement portion 28 are engaged with each other in the connected state to allow communication between the loading lumen 22 and the sheath lumen 42.

In the delivery system 200, since the second engagement portion 28 including the plurality of engagement claws 28a provided in the delivery catheter 40 is engaged with the groove portion 48a, which is the first engagement portion 48 provided in the embolic device loading catheter 20, the embolic device loading catheter 20 is attachable to and detachable from the delivery catheter 40. Therefore, after the embolic device 10 is pushed out into the aneurysm by the push-out operation, by releasing an engagement state between the first engagement portion 48 and the second engagement portion 28, it is possible to rather easily restart an indwelling operation of the embolic device 10 including the push-out operation by connecting (mounting) another embolic device loading catheter 20 in which the embolic device 10 has been loaded or reloading the embolic device 10 into the embolic device loading catheter 20 which has been detached.

The delivery system 200 according to the present embodiment may be configured such that, preferably, the communication passage 43a of the sheath hub 43 of the delivery catheter 40 includes the communication distal portion 43b having the inner diameter substantially equal to the inner diameter of the sheath lumen 42, and the communicating enlarged-diameter portion 43c extending from the communication distal portion 43b in the proximal direction and having a diameter larger than that of the communication distal portion 43b, and the distal connection portion 27 of the catheter body 21 includes the distal end of the loading lumen 22 and includes the insertion portion 27a to be inserted into the communicating enlarged-diameter portion 43c of the sheath hub 43 in the connected state.

Since the insertion portion 27a is inserted such that the loading lumen 22 and the sheath lumen 42 are aligned in the axial direction in the connected state between the embolic device loading catheter 20 and the delivery catheter 40, the embolic device 10 is pushed out to the sheath lumen 42 from the loading lumen 22 without being exposed to the outside through the sheath hub 43.

The delivery system 200 according to the present embodiment may be configured such that, preferably, the communicating enlarged-diameter portion 43c includes the tapered portion 43d enlarged in diameter in the proximal direction, and the insertion portion 27a of the distal connection portion 27 includes the distal contact portion 27b that comes into contact with the inner surface of the tapered portion 43d such that the axis of the loading lumen 22 and the axis of the sheath lumen 42 of the delivery catheter 40 are aligned in the connected state.

When the insertion portion 27a is inserted into the communicating enlarged-diameter portion 43c, if the distal contact portion 27b comes into contact with the tapered portion 43d, the loading lumen 22 and the sheath lumen 42 communicate with each other so as not to intersect with other spaces including the space of the communicating enlarged-diameter portion 43c. Therefore, when the embolic device 10 is pushed out from the embolic device loading catheter 20 into the delivery catheter 40, the embolic device 10 is prevented from being damaged (for example, bent or crushed on the distal side) due to abutment of the embolic device 10 against the inner wall surface of the sheath hub 43, is prevented from being erroneously inserted into other spaces within the sheath hub 43 (for example, erroneously entering into the tube 44 connected to the sheath hub 43), and is reliably delivered into the aneurysm.

The delivery system 200 according to the present embodiment may be configured such that, preferably, the insertion portion 27a includes the bending portion 27c that is gradually bent away from the axial direction of the loading lumen 22 toward the distal end of the loading lumen 22 (distal contact portion 27b), and when the insertion portion 27a is inserted into the sheath hub 43, the bent shape of the bending portion 27c is corrected, and in the connected state, the distal contact portion 27b and the inner surface of the tapered portion 43d come into contact with each other, and the axis of the loading lumen 22 and the axis of the sheath lumen 42 of the delivery catheter 40 are aligned.

By providing the bending portion 27c in at least a part of the insertion portion 27a, it is possible to prevent the embolic device 10 loaded in the loading lumen 22 from erroneously falling off from the distal end of the distal connection portion 27. When the bending portion 27c is inserted into the sheath hub 43, the bent shape of the bending portion 27c is corrected to a substantially straight state. Therefore, since the axis of the loading lumen 22 and the axis of the sheath lumen 42 are aligned in the connected state between the embolic device loading catheter 20 and the delivery catheter 40, the insertion of the embolic device 10 is not hindered.

The delivery system 200 according to the present embodiment may be configured such that, preferably, the insertion portion 27a of the distal connection portion 27 is inserted into the communicating enlarged-diameter portion 43c such that the distal end of the loading lumen 22 is adjacent to the communication distal portion 43b in the connected state.

Since the insertion portion 27a is inserted into the communicating enlarged-diameter portion 43c such that the distal end of the loading lumen 22 is adjacent to the communication distal portion 43b, the embolic device 10 pushed out from the embolic device loading catheter 20 to the delivery catheter 40 is prevented from being damaged by abutting against the inner wall surface of the sheath hub 43 or from being erroneously inserted into other spaces within the sheath hub 43.

In the delivery system 200 according to the present embodiment, preferably, the catheter body 21 of the embolic device loading catheter 20 may be configured to have rigidity higher than that of the sheath 41 of the delivery catheter 40.

In the delivery system 200, since the embolic device loading catheter 20 is not used by being inserted into a sheath of the delivery catheter 40 as in the direct insertion method, the embolic device loading catheter 20 does not require flexibility to follow the bent shape of the body lumen during insertion, and the rigidity of the catheter body 21 can be made higher than that of the sheath 41. Therefore, the embolic device 10 is prevented from being damaged during packaging or unpacking.

The embolic device delivery medical system 300 according to the present embodiment includes: the delivery system 200; and the elongated insertion assisting member 50 to be assembled into the sheath lumen 42 of the delivery catheter 40 in order to assist delivery of the delivery catheter 40 into the aneurysm. The insertion assisting member 50 includes the guide wire lumen 52 that penetrates from a distal end to a proximal end and has a diameter smaller than that of the sheath lumen 42 of the delivery catheter 40.

With such a configuration, when the delivery catheter 40 is to be inserted into the body lumen, since the insertion assisting member 50 is assembled, the delivery catheter 40 can be inserted into the body lumen relatively smoothly.

The detailed description above describes embodiments of a medical instrument set, a delivery system, and an embolic device delivery medical system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical instrument set for delivering an embolic device into an aneurysm, the medical instrument set comprising:
an embolic device loading catheter that includes a catheter body including a loading lumen configured to be loaded with the embolic device and having an open distal end, and a proximal hub including an insertion passage communicating with a proximal end of the loading lumen;
a delivery pusher that includes an elongated pusher body insertable into the loading lumen through the insertion passage of the proximal hub of the embolic device loading catheter;
the embolic device loading catheter including a distal connection portion configured to be attachable to and detachable from a sheath hub of a delivery catheter at a distal portion of the catheter body; and
the pusher body having a body length longer than a distance from a proximal end of the insertion passage of the proximal hub of the embolic device loading catheter to the distal end opening of the sheath of the delivery catheter in a connected state where the distal connection portion of the catheter body is connected to the sheath hub of the delivery catheter, and when the pusher body is inserted from the insertion passage of the proximal hub in the connected state, the embolic device is configured to pass through the sheath lumen of the delivery catheter and to be pushed out from the distal end opening of the delivery catheter into the aneurysm.

2. The medical instrument set according to claim 1, wherein an inner diameter of the loading lumen is approximately the same as an inner diameter of the sheath lumen.

3. The medical instrument set according to claim 1, wherein the distal connection portion of the catheter body includes an insertion portion inserted into the sheath hub of the delivery catheter in the connected state.

4. The medical instrument set according to claim 1, wherein
the delivery pusher includes a handle portion provided on a proximal side of the pusher body; and
a maximum outer diameter of the handle portion is larger than an inner diameter of the insertion passage of the proximal hub.

5. A delivery system comprising:
the medical instrument set according to claim 1;
the delivery catheter, the delivery catheter including a sheath that includes a sheath lumen and a distal end opening that communicates with the sheath lumen, and a sheath hub that includes a communication passage communicating with a proximal end of the sheath lumen and is provided on a proximal side of the sheath; and
wherein in the connected state, the embolic device is configured to pass through the sheath lumen of the delivery catheter by the delivery pusher inserted from the insertion passage of the proximal hub, and is configured to be pushed out from the distal end opening of the delivery catheter into the aneurysm.

6. The delivery system according to claim 5, wherein
the delivery catheter includes a first engagement portion including a groove portion provided on a proximal side of the sheath hub;
the embolic device loading catheter includes a second engagement portion having an engagement claw that engages with the first engagement portion at the distal connection portion; and
in the connected state, the first engagement portion and the second engagement portion are configured to engage with each other to allow the loading lumen to communicate with the sheath lumen.

7. The delivery system according to claim 5, wherein
the communication passage of the sheath hub of the delivery catheter includes a communication distal portion having an inner diameter substantially equal to an inner diameter of the sheath lumen, and a communicating enlarged-diameter portion extending from the communication distal portion in a proximal direction and having a diameter larger than that of the communication distal portion; and
the distal connection portion of the catheter body includes an insertion portion that includes a distal end of the loading lumen, and the insertion portion is configured to be inserted into the communicating enlarged-diameter portion of the sheath hub in the connected state.

8. The delivery system according to claim 7, wherein
the communicating enlarged-diameter portion has a tapered portion whose diameter is enlarged in the proximal direction; and
the insertion portion of the distal connection portion includes a distal contact portion that comes into contact with an inner surface of the tapered portion such that an axis of the loading lumen and an axis of the sheath lumen of the delivery catheter are aligned in the connected state.

9. The delivery system according to claim 8, wherein
the insertion portion includes a bending portion that is bent away from an axial direction of the loading lumen toward the distal end of the loading lumen; and
the insertion portion is configured such that a bending shape of the bending portion is corrected when the insertion portion is inserted into the sheath hub, and in the connected state, the distal contact portion and the inner surface of the tapered portion is configured to come into contact with each other, and the axis of the loading lumen and the axis of the sheath lumen of the delivery catheter are configured to be aligned.

10. The delivery system according to claim 7, wherein the insertion portion of the distal connection portion is configured to be inserted into the communicating enlarged-diameter portion such that the distal end of the loading lumen is adjacent to the communication distal portion in the connected state.

11. The delivery system according to claim 5, wherein the catheter body of the embolic device loading catheter has a rigidity that is greater than a rigidity of the sheath of the delivery catheter.

12. An embolic device delivery medical system comprising:
a delivery catheter, the delivery catheter including a sheath that includes a sheath lumen and a distal end opening that communicates with the sheath lumen, and a sheath hub that includes a communication passage communicating with a proximal end of the sheath lumen and is provided on a proximal side of the sheath;
an embolic device loading catheter that includes a catheter body including a loading lumen configured to be loaded with the embolic device and having an open distal end, and a proximal hub including an insertion passage communicating with a proximal end of the loading lumen;
a delivery pusher that includes an elongated pusher body insertable into the loading lumen through the insertion passage of the proximal hub of the embolic device loading catheter;
the embolic device loading catheter including a distal connection portion configured to be attachable to and detachable from a sheath hub of a delivery catheter at a distal portion of the catheter body;
the pusher body having a body length longer than a distance from a proximal end of the insertion passage of the proximal hub to the distal end opening of the sheath of the delivery catheter in a connected state where the distal connection portion of the catheter body is connected to the sheath hub of the delivery catheter, and when the pusher body is inserted from the insertion passage of the proximal hub in the connected state, the embolic device is configured to pass through the sheath lumen of the delivery catheter and to be pushed out from the distal end opening of the delivery catheter into the aneurysm; and
an elongated insertion assisting member configured to be assembled into the sheath lumen of the delivery catheter in order to assist delivery of the delivery catheter into the aneurysm, and wherein the insertion assisting member includes a guide wire lumen that penetrates from a distal end to a proximal end and has a diameter smaller than that of the sheath lumen of the delivery catheter.

13. The embolic device delivery medical system according to claim 12, wherein in the connected state, the embolic device is configured to pass through the sheath lumen of the delivery catheter by the delivery pusher inserted from the insertion passage of the proximal hub, and the embolic device is configured to be pushed out from the distal end opening of the delivery catheter into the aneurysm.

14. The embolic device delivery medical system according to claim 12, further comprising:
the embolic device, the embolic device configured to be indwellable in the aneurysm and configured to, when the embolic device is indwelled in the aneurysm, absorb liquid in the aneurysm and expand.

15. A method comprising:
introducing a distal portion of a delivery catheter into a living body and positioning the distal end of the delivery catheter in an aneurysm in the living body, the delivery catheter including a sheath that includes a sheath lumen and a distal end opening that communicates with the sheath lumen, and a sheath hub that includes a communication passage communicating with a proximal end of the sheath lumen and is provided on a proximal side of the sheath;
connecting a distal portion of an embolic device loading catheter to the proximal end of the delivery catheter, the embolic device loading catheter including a catheter body including a loading lumen loaded with an embolic device and having an open distal end, and a proximal hub including an insertion passage communicating with a proximal end of the loading lumen;
introducing a distal end of a delivery pusher into the embolic device loading catheter by way of the proximal end of the loading lumen, the delivery pusher including an elongated pusher body insertable into the loading lumen through the insertion passage of the proximal hub of the embolic device loading catheter, and wherein the pusher body has a body length longer than a distance from a proximal end of the insertion passage of the proximal hub of the embolic device loading catheter to the distal end opening of the sheath of the delivery catheter when the distal portion of the catheter body is connected to the proximal end of the delivery catheter; and
moving the delivery pusher in a forward direction to push the embolic device out of the embolic loading catheter into the sheath lumen of the delivery catheter and out of the sheath lumen of the delivery catheter into the aneurysm.

16. The method according to claim 15, further comprising:
detaching the embolic loading catheter from the delivery catheter after the embolic device has been pushed out of the sheath lumen of the delivery catheter into the aneurysm.

17. The method according to claim 16, further comprising:
repeating the connecting of the distal portion of the embolic device loading catheter to the proximal end of the delivery catheter, the introduction of the distal end of the delivery pusher into the embolic device loading catheter by way of the proximal end of the embolic device loading catheter, and the moving of the delivery in the forward direction to push the embolic device out of the embolic device loading catheter into the sheath lumen of the delivery catheter and out of the sheath lumen of the delivery catheter into the aneurysm until a desired amount of embolic device has been loaded into the aneurysm.

18. The method according to claim 15, further comprising:
assisting delivery of the delivery catheter into the aneurysm with an elongated insertion assisting member, the elongated insertion assisting member including a guide wire lumen that penetrates from a distal end to a proximal end and has a diameter smaller than that of the sheath lumen of the delivery catheter

19. The method according to claim 15, further comprising:
indwelling an introducer into the living body; and
introducing the distal portion of the delivery catheter into the living body via the introducer.

20. The method according to claim 19, further comprising:
removing the delivery catheter after a desired amount of embolic device has been loaded into the aneurysm; and
performing an expansion of a stent graft by a balloon or an imaging operation of the aneurysm.
